(19) United States
(12) Patent Application Publication
(10) Pub. No.: US 2025/0043131 A1
(43) Pub. Date: Feb. 6, 2025

(54) AQUEOUS FIBROIN SOLUTION AND PRODUCTION METHOD THEREOF, AND ARTICLE COMPRISING FIBROIN

(71) Applicants: CANON KABUSHIKI KAISHA, Tokyo (JP); CANON VIRGINIA, INC., Newport News, VA (US)

(72) Inventors: KEI INOUE, Kanagawa (JP); YUMA KOBAYASHI, Kanagawa (JP); KEIGO MIZUSAWA, Tokyo (JP); WAKA HASEGAWA, Tokyo (JP); Jun Shirakawa, Saitama (JP)

(21) Appl. No.: 18/900,302

(22) Filed: Sep. 27, 2024

Related U.S. Application Data
(63) Continuation of application No. PCT/JP2023/011765, filed on Mar. 24, 2023.

(30) Foreign Application Priority Data
Mar. 31, 2022 (JP) 2022-060225
Mar. 20, 2023 (JP) 2023-044097

(51) Int. Cl. C08L 89/00; C08K 5/19; C08K 5/3432; C08K 5/3445

(52) U.S. Cl. CPC C08L 89/00; C08K 5/19; C08K 5/3432; C08K 5/3445

(57) ABSTRACT

Provided are a fibroin aqueous solution excellent in storage stability and also excellent in foamability and a method of producing the same. The fibroin aqueous solution includes fibroin and a compound represented by the general formula (1) or (2).

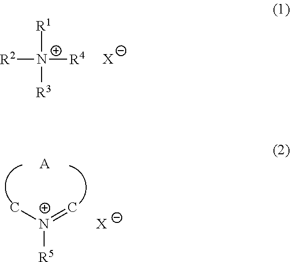

### CROSS-REFERENCE TO RELATED APPLICATIONS

This application is a Continuation of International Patent Application No. PCT/JP2023/011765, filed Mar. 24, 2023, which claims the benefit of Japanese Patent Application No. 2022-060225, filed Mar. 31, 2022, and Japanese Patent Application No. 2023-044097, filed Mar. 20, 2023, all of which are hereby incorporated by reference herein in their entirety.

### BACKGROUND

### Field

The present invention relates to a fibroin aqueous solution and a method of producing the same, and an article including fibroin.

### Description of the Related Art

Fibroin that is a main component of silk thread has high biocompatibility and has been used for surgical suture thread and the like for a long time, and is known to be highly safe. Recently, the application of various types of foam, such as gel, a sponge, a film, and a nonwoven fabric, prepared from a fibroin aqueous solution to medical fields, such as a cell culture scaffold, a wound dressing, an artificial skin, and an artificial bone has been actively investigated.

As a method of producing the fibroin aqueous solution, a method involving refining an unrefined fibroin raw material, such as a cocoon or raw silk, dissolving the resultant in a neutral salt aqueous solution having a high concentration, and then desalting the solution by dialysis, ultrafiltration, or the like is generally used. However, the fibroin aqueous solution obtained in this manner easily undergoes a change in crystal structure due to external stimulation, a change with time, and the like, resulting in gelling. Thus, there has been a demand for a fibroin aqueous solution having high storage stability that does not undergo a change in crystal structure.

Various additives have been investigated in order to enhance the storage stability of the fibroin aqueous solution. For example, in Japanese Patent Application Laid-Open No. H7-90182 and Japanese Patent Application Laid-Open No. 2008-169171, there has been proposed that urea or thiourea be added as a protein denaturant. In addition, in Japanese Patent Application Laid-Open No. 2015-140328, there has been proposed that the storage stability of the fibroin aqueous solution be improved by adding a guanidino group-containing compound such as arginine.

The above-mentioned additives each have an action of strongly denaturing a protein, to thereby prevent the gelling of an aqueous solution to improve its storage stability. Meanwhile, when an attempt is made to form various types of foam, such as gel and a sponge, from an aqueous solution, there is a problem in that crystallization does not easily occur and a long time period is taken for the formation.

In view of the foregoing, an object of the present invention is to provide a fibroin aqueous solution that is excellent in storage stability and can be easily formed into various types of foam, and a method of producing the same.

### SUMMARY

According to an embodiment of the present invention, there is provided a fibroin aqueous solution including: fibroin; and a compound represented by the following general formula (1) or (2):

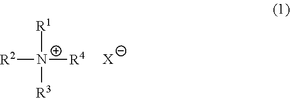

in the general formula (1),
R¹ to R⁴ each independently represent
   a hydrogen atom,
   an aliphatic hydrocarbon group having 1 or more and 8 or less carbon atoms, which may have a substituent, or
   an aryl group or an aralkyl group having 6 or more and 10 or less carbon atoms, which may have a substituent,
provided that at least one of R¹ to R⁴ does not represent a hydrogen atom, and
X⁻ represents an anion;

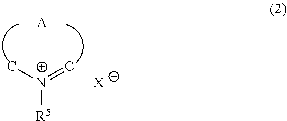

in the general formula (2),
C—N═C and A form a ring structure together,
R⁵ represents
   a hydrogen atom,
   an aliphatic hydrocarbon group having 1 or more and 8 or less carbon atoms, which may have a substituent, or
   an aryl group or an aralkyl group having 6 or more and 10 or less carbon atoms, which may have a substituent, and
X⁻ represents an anion.

An article according to the present invention includes: fibroin; and a compound represented by the following general formula (1) or (2):

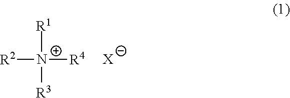

in the general formula (1),
R¹ to R⁴ each independently represent
   a hydrogen atom,
   an aliphatic hydrocarbon group having 1 or more and 8 or less carbon atoms, which may have a substituent, or
   an aryl group or an aralkyl group having 6 or more and 10 or less carbon atoms, which may have a substituent,
provided that at least one of R¹ to R⁴ does not represent a hydrogen atom, and
X⁻ represents an anion;

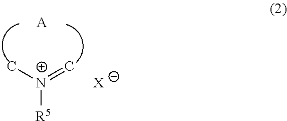

in the general formula (2),
C—N═C and A form a ring structure together,
R⁵ represents
   a hydrogen atom,
   an aliphatic hydrocarbon group having 1 or more and 8 or less carbon atoms, which may have a substituent, or
   an aryl group or an aralkyl group having 6 or more and 10 or less carbon atoms, which may have a substituent, and
X⁻ represents an anion.

Further features of the present invention will become apparent from the following description of exemplary embodiments.

### DETAILED DESCRIPTION

Fibroin used in this embodiment is a fibrous protein derived from an organism classified into Lepidoptera, Hymenoptera, or Araneae, and may be obtained by a genetic recombination technology. Fibroin derived from a cocoon of a domesticated silkworm is preferred from the viewpoint of the ease of availability of a raw material.

The fibroin used in this embodiment may prepared from a raw material, such as a cocoon of a domesticated silkworm, cocoon thread, processed cocoon thread (e.g., silk thread), or residual thread from processed cocoon thread. In other words, fibroin derived from silk, such as a cocoon of a domesticated silkworm, cocoon thread, processed cocoon thread (e.g., silk thread), or residual thread from processed cocoon thread, may be used as the fibroin in this embodiment. The fibroin may be obtained by removing sericin from such raw material through use of a known refining method. The resultant fibroin may be turned into an aqueous solution by dissolving the resultant fibroin in an aqueous solution of lithium bromide or calcium chloride having a high concentration, and then desalting the resultant by a method, such as dialysis or ultrafiltration using a semipermeable membrane. The resultant aqueous solution is unstable, and forms gel to be solidified when left to stand at normal temperature. Thus, the aqueous solution is preferably stored under refrigeration at about 4° C.

The molecular weight of the fibroin used in this embodiment is not particularly limited, but fibroin having a higher molecular weight of 100,000 or more exhibits higher effects. In general, when the molecular weight of the fibroin becomes higher, the storage stability of the aqueous solution thereof tends to become lower. However, when the molecular weight is 100,000 or less, sufficient storage stability is exhibited in many cases even without the addition of the additive of the present invention. When the molecular weight of the fibroin is more than 100,000, the storage stability is decreased, and it is required to add the additive for long-term storage. In addition, when the molecular weight is 100,000 or less, the dynamic characteristics of various types of foam formed from the aqueous solution are decreased, and hence such molecular weight is not preferred in some applications of the foam.

The fibroin used in this embodiment has a molecular weight of preferably 100,000 or more, more preferably 150,000 or more. In addition, the molecular weight of the fibroin derived from the domesticated silkworm is 350,000, and hence the upper limit of the molecular weight of the fibroin used in this embodiment is substantially 350,000.

The molecular weight of the fibroin may be controlled by a temperature and a time at the time of refining. The fibroin derived from the domesticated silkworm has a molecular weight of about 350,000, but fibroin having a desired molecular weight may be obtained by changing the refining temperature and time. In general, when the refining temperature becomes higher or the refining time becomes longer, fibroin having a lower molecular weight is obtained.

The fibroin aqueous solution according to this embodiment is characterized by including a compound represented by the general formula (1) or (2).

Through incorporation of the above-mentioned compound, an aqueous solution that is excellent in storage stability and is easily formed into various types of foam is obtained.

It is considered that urea and a guanidino group-containing compound, which are general protein denaturants, are selectively bonded to peptide chains to inhibit the formation of a hydrogen bond between the peptide chains, to thereby suppress gelling. Such compound effectively acts in order to enhance the stability of the aqueous solution. However, in the case where various types of foam are produced through use of the aqueous solution, when such compound is used, the formation of a hydrogen bond is inhibited, and hence a long period of time is taken for the foam formation.

Meanwhile, although the mechanism of action of the compound according to this embodiment is not clear, it is conceived that the compound forms an ion pair with a carboxyl group derived from a glutamic acid or aspartic acid residue and suppresses the approach between peptides through a steric or electrostatic action. The aqueous solution is stabilized by such mechanism. Thus, it is conceived that, when external stimulation is applied at the time of the formation of various types of foam, peptide chains easily form a hydrogen bond, and the formation of various types of foam also becomes easy.

In the general formula (1), R¹ to R⁴ each independently represent a hydrogen atom, an aliphatic hydrocarbon group having 1 or more and 8 or less carbon atoms, which may have a substituent, or an aryl group or an aralkyl group having 6 or more and 10 or less carbon atoms, which may have a substituent, provided that at least one of R¹ to R⁴ does not represent a hydrogen atom, and X⁻ represents an anion. In addition, in the general formula (2), C—N═C and A form a ring structure together, R⁵ represents a hydrogen atom, an aliphatic hydrocarbon group having 1 or more and 8 or less carbon atoms, which may have a substituent, or an aryl group or an aralkyl group having 6 or more and 10 or less carbon atoms, which may have a substituent, and X⁻ represents an anion.

The compound in which the number of carbon atoms is more than the foregoing has high hydrophobicity, and hence a hydrophobic interaction preferentially acts over ion pair formation with peptide chains. Thus, it becomes difficult to form foam, though a stabilizing effect on the aqueous solution is obtained.

Examples of the aliphatic hydrocarbon group having 1 or more and 8 or less carbon atoms in the R¹, R², R³, R⁴, or R⁵ include a methyl group, an ethyl group, a n-propyl group, an isopropyl group, a n-butyl group, a n-octyl group, and a 2-ethylhexyl group, and those aliphatic hydrocarbon groups may be bonded to each other to form a ring. In addition, examples of the substituent include a hydroxy group, an amino group, an alkoxycarbonyl group, and a carbamoyl group.

Examples of the aryl group or aralkyl group having 6 or more and 10 or less carbon atoms in the R¹, R², R³, R⁴, or R⁵ include: aryl groups, such as a phenyl group, a 1-naphthyl group, and a 2-naphthyl group; and aralkyl groups, such as a methyl group, an ethyl group, a n-propyl group, an isopropyl group, and a n-butyl group substituted with the aryl groups. In addition, examples of the substituent include an alkyl group, a hydroxy group, an amino group, an alkoxycarbonyl group, and a carbamoyl group.

In the general formula (2), C—N═C and A form a ring structure together. For example, C—N═C and A can form pyridine or imidazole together. A cationic moiety in the general formula (2) is preferably a heterocyclic aromatic compound. Examples thereof include a 1-alkylpyridinium cation and a 1,3-dialkylimidazolium cation.

In each of the general formulae (1) and (2), X⁻ may represent a halide ion, a hydroxide ion, or a carboxylate anion. Examples of the halide ion include a fluoride ion, a chloride ion, a bromide ion, and an iodide ion.

Examples of the carboxylate anion include an acetate anion, a propionate anion, a benzoate anion, a tartrate anion, and a bitartrate anion.

The compounds represented by the general formulae (1) and (2) are not particularly limited as long as the compounds have the above-mentioned substituents. However, the compounds are preferably water-soluble from the viewpoint that the compounds are each added to a fibroin aqueous solution to improve its storage stability. The compounds each preferably exhibit a solubility of 0.01 mass % or more with respect to water, and more preferably have a solubility of 0.1 mass % or more with respect to water. When the water solubility is less than 0.01 mass %, a sufficient improving effect on stability is not obtained.

The compounds represented by the general formulae (1) and (2) are preferably salts of a tetraalkylammonium, a choline derivative, and a glycine derivative in consideration of the ease of availability, and the influence of residual components at the time of the formation of various types of foam on biocompatibility and the like. Examples thereof include a halide, a hydroxide, and a carboxylate of any one selected from a tetraalkylammonium, choline, a choline derivative, glycine, and a glycine derivative, and include a tetraalkylammonium halide, and a halide, a hydroxide, and a carboxylate of any one selected from choline, a choline derivative, glycine, and a glycine derivative.

Specific examples of the above-mentioned compounds include tetramethylammonium chloride, tetramethylammonium acetate, tetraethylammonium bromide, tetraethylammonium hydroxide, tetrapropylammonium chloride, tetrabutylammonium chloride, triethylmethylammonium chloride, trimethylphenylammonium chloride, choline, choline chloride, choline bitartrate, trimethylamine hydrochloride, triethanolamine hydrochloride, dibutylamine hydrochloride, glycine ethyl ester hydrochloride, glycinamide hydrochloride, methylpyridinium chloride, and 1,3-dimethylimidazolium chloride. Further, preferred specific examples thereof include tetramethylammonium chloride, tetramethylammonium bromide, tetraethylammonium chloride, tetrapropylammonium chloride, tetrabutylammonium chloride, triethylmethylammonium chloride, choline chloride, choline bitartrate, glycine methyl ester hydrochloride, glycine ethyl ester hydrochloride, and glycinamide hydrochloride.

The addition amount of the compounds represented by the general formulae (1) and (2) in the fibroin aqueous solution of this embodiment is not particularly limited, and is appropriately adjusted in accordance with desired characteristics. As a general addition amount, 0.1 mass % or more and 50 mass % or less with respect to the mass of the fibroin dissolved in the aqueous solution is appropriate. When the addition amount is less than 0.1 mass %, sufficient storage stability is not obtained. Meanwhile, when the addition amount is more than 50 mass %, it may be difficult to form the fibroin aqueous solution into various types of foam, though high storage stability is obtained.

The concentration of the fibroin in the fibroin aqueous solution according to this embodiment is not particularly limited, but the effects are particularly high when the concentration of the fibroin is 5 mass % or more and 40 mass % or less with respect to the total mass of the aqueous solution. In general, when the concentration of the fibroin becomes higher, the storage stability of the aqueous solution thereof tends to become lower. However, when the concentration of the fibroin is less than 5 mass %, sufficient storage stability is exhibited in many cases even without the addition of the additive of the present invention. Meanwhile, when the concentration of the fibroin is more than 40 mass %, it may be difficult to prepare the aqueous solution itself, and sufficient storage stability may not be obtained even when the additive of the present invention is added.

The fibroin aqueous solution according to this embodiment may be produced through: a refining step of removing sericin from a fibroin raw material; a neutral salt dissolving step of dissolving the refined fibroin raw material in a neutral salt aqueous solution to provide a fibroin-neutral salt aqueous solution; a desalting step of desalting the fibroin-neutral salt aqueous solution to provide a fibroin aqueous solution; an additive adding step of adding the compound represented by the general formula (1) or (2); and a concentration adjusting step of adjusting a concentration of the aqueous solution.

In the refining step, the neutral salt dissolving step, and the desalting step, known methods may be used, and these steps are not particularly limited.

The additive adding step may be performed between any of the above-mentioned production steps, but is preferably performed after the desalting step or the concentration adjusting step from the viewpoint of managing the concentration of the additive. As a method of adding the additive, a method involving adding the additive directly to the fibroin aqueous solution or a method involving adding the additive as an aqueous solution after dissolving the additive in water may be appropriately selected, but the method involving adding the additive as an aqueous solution is preferred from the viewpoint of eliminating the concentration unevenness of the additive. After the additive is added, stirring is preferably performed in order to make the concentration of the additive in the aqueous solution uniform. The fibroin aqueous solution may form gel when a strong shear force is applied thereto, and hence it is required to select a stirring method with a low shear force.

The concentration adjusting step is preferably performed after the desalting step or the additive adding step. In the concentration adjusting step, the fibroin aqueous solution may be diluted or concentrated so as to achieve an intended fibroin concentration.

When the aqueous solution is diluted, it is preferred that, after water is added so as to achieve an intended concentration, the aqueous solution be stirred so that the entire aqueous solution achieves a uniform concentration. A stirring method is not particularly limited, but for the same reason as described above, a stirring method with a low shear force is preferred.

When the aqueous solution is concentrated, a known concentration method may be used. Although the concentration method is not particularly limited, a method involving applying less heat and a lower shear force is preferred in order to reduce the deterioration and denaturation of the fibroin aqueous solution. For example, methods, such as ultrafiltration or dialysis using a semipermeable membrane, centrifugal concentration, and concentration under low temperature and reduced pressure, are particularly preferred.

The production of the fibroin aqueous solution may further include a step of removing insoluble matter generated in the aqueous solution for the purpose of further improving the storage stability. When the insoluble matter in the aqueous solution is removed, the generation of gel can be suppressed, and the storage stability of the aqueous solution can be further improved.

The step of removing insoluble matter preferably includes: a heating step of heating the fibroin aqueous solution at 65° C. or more and 110° C. or less for a time period of 30 minutes or more and 60 minutes or less; a cooling step of rapidly cooling the fibroin aqueous solution heated in the heating step to 15° C. or less; and a microfiltration step of filtering the fibroin aqueous solution cooled in the cooling step through use of a microfiltration membrane.

In the heating step, the fibroin aqueous solution obtained in the above-mentioned desalting step is heated at 65° C. or more and 110° C. or less for a time period of 30 minutes or more and 60 minutes or less.

When the heating temperature is less than 65° C., components that are easily insolubilized to form aggregates are not sufficiently precipitated in the cooling step described later, and aggregates may not be sufficiently generated in the cooling step. In addition, when the heating temperature is more than 110° C., thermal denaturation of the fibroin may proceed to cause quality degradation. The heating temperature is preferably 65° C. or more and 105° C. or less, more preferably 70° C. or more and 105° C. or less, still more preferably 85° C. or more and 95° C. or less. When the heating temperature falls within those ranges, aggregates are sufficiently generated in the cooling step and can be sufficiently removed in the microfiltration step, with the result that the storage stability of the resultant fibroin aqueous solution becomes more excellent.

When the heating time is less than 30 minutes, components that are easily insolubilized to form aggregates are not sufficiently precipitated in the cooling step described later, and aggregates may not be sufficiently generated in the cooling step. In addition, microorganisms, such as bacteria, which may cause quality degradation during long-term storage, may not be sufficiently sterilized. Meanwhile, when the heating time is more than 60 minutes, thermal denaturation and gelling of the fibroin may proceed to cause quality degradation. The heating time is preferably 40 minutes or more and 60 minutes or less, more preferably 45 minutes or more and 60 minutes or less. When the heating time falls within those ranges, aggregates are sufficiently generated in the cooling step and can be sufficiently removed in the microfiltration step, with the result that the storage stability of the resultant fibroin aqueous solution becomes more excellent.

A heating method is not particularly limited, and a heating method known in the related art may be used. Specific examples thereof include an autoclave, a heater, and a microwave oven.

In the cooling step, the fibroin aqueous solution heated in the above-mentioned heating step is rapidly cooled to 15° C. or less.

The advantages of the rapid cooling include: an advantage from a production efficiency aspect in which the speed of settling separation is increased because aggregates suspended in the liquid are rapidly aggregated by cooling; and an advantage from sanitary and quality aspects in which a time period during which the temperature stays in a range suitable for growth of germs is minimized to suppress the growth of germs.

The cooling temperature is not particularly limited as long as the cooling temperature is 15° C. or less and does not cause freezing, but is preferably 0° C. or more and 10° C. or less, more preferably 3° C. or more and 8° C. or less. When the cooling temperature falls within those ranges, insoluble components or components that are easily insolubilized in the heated fibroin aqueous solution easily form aggregates, and hence separation and removal from the fibroin aqueous solution can be made more reliable.

In the present invention, the rapid cooling generally refers to cooling at a cooling rate of 0.2° C./sec or more. The cooling rate at the time of the cooling is preferably 0.3° C./sec or more and 0.8° C./sec or less, more preferably 0.4° C./sec or more and 0.7° C./sec or less, still more preferably 0.5° C./sec or more and 0.6° C./sec or less.

When the cooling rate falls within those ranges, insoluble components or components that are easily insolubilized in the heated fibroin aqueous solution easily form aggregates, and hence separation and removal from the fibroin aqueous solution can be made more reliable.

A rapid cooling method is not particularly limited, but examples thereof include methods using cooling means, such as cold water, ice, ice water, dry ice, dry ice and ethanol, and a rapid cooler. Of those, ice water is preferably used because the ice water is easy to handle.

In the microfiltration step, the fibroin aqueous solution cooled in the above-mentioned cooling step is filtered through use of a microfiltration membrane.

Examples of the microfiltration membrane include microfiltration membranes formed of materials, such as cellulose acetate, an aromatic polyamide, polyvinyl alcohol, polysulfone, polyvinylidene fluoride, polyethylene, polyacrylonitrile, a ceramic, polypropylene, polycarbonate, and a fluororesin.

The pore diameter of the microfiltration membrane is not particularly limited, but is preferably 0.40 m or more and 1.2 m or less, more preferably 0.45 m or more and 1.0 m or less, still more preferably 0.60 m or more and 1.0 m or less. When the pore diameter falls within those ranges, aggregates in the fibroin aqueous solution can be removed more quickly and sufficiently. The shape of the microfiltration membrane is not particularly limited, but examples thereof include a flat membrane, a tubular membrane, a spiral membrane, and a hollow fiber membrane. Of those, a hollow fiber membrane is preferred because the hollow fiber membrane has low energy cost, can be used at a relatively low pressure, and has a low risk of denaturation of protein components in a liquid by a pressure.

Various types of foam, such as gel, a sponge, a film, and a nonwoven fabric, may be produced through use of the fibroin aqueous solution of this embodiment. The foam produced through use of the fibroin aqueous solution of this embodiment may be called an article including fibroin. Through incorporation of the compound represented by the general formula (1) or (2), the above-mentioned foam can be formed more quickly as compared to the case in which the additives, such as urea and guanidine hydrochloride, are used. The fibroin aqueous solution of this embodiment is more effective in foam prepared in an aqueous solution, such as gel or a sponge. Production methods therefor are not particularly limited. Any known methods may be used, and any external stimulation that accelerates the crystallization (β-sheet formation) of fibroin may be used. A gel production method that may be used is, for example, a method using a pH change with hydrochloric acid, a method using a chemical substance with a gelling accelerator, a method using a shear force with strong stirring, or a method using the application of an electric field. A sponge production method that may be used is, for example, a method using a porogen, such as salt or sugar, or a method involving freeze-drying an aqueous solution and then annealing the resultant with heat or a solvent. In addition, a powder production method that may be used is, for example, a spray drying method or a freeze-drying method, and a film production method that may be used is, for example, a casting method.

The article according to this embodiment includes fibroin and the compound represented by the general formula (1) or (2). The article according to this embodiment may be a solid substance having any one of, for example, powder, film, and sponge shapes, or may have a gel shape. Alternatively, the article according to this embodiment may be a molded body that is molded with a mold.

### EXAMPLES

The present invention is described in more detail below by way of Examples and Comparative Examples. However, the present invention is by no means limited to Examples below, and various modifications are possible without departing from the gist of the present invention. In the description of the amounts of components, “part(s)” and “%” are by mass unless otherwise specified.

### <Method of Measuring Concentration of Fibroin Aqueous Solution>

The concentration of a fibroin aqueous solution prepared in each of Examples and Comparative Examples described below was calculated as follows: 0.5 mL of the fibroin aqueous solution was loaded into a tared glass container; the fibroin aqueous solution was dried in an oven adjusted to 60° C. for 2 hours or more; and its solid content concentration was calculated from a change in weight before and after the drying.

### Method of Measuring Molecular Weight

The molecular weight of the fibroin aqueous solution prepared in each of Examples and Comparative Examples described below was measured with a microchip electrophoresis device Agilent 2100 Bioanalyzer Electrophoresis System (manufactured by Agilent Technologies) under the following conditions.
Microchip, separation matrix, fluorescent dye, electrophoresis buffer, and molecular weight standard ladder: Agilent Protein 230 kit
Control sample: bovine serum albumin lyophilized powder, >96% (agarose gel electrophoresis) (manufactured by Sigma-Aldrich Co., LLC, molecular weight: 66.5 kDa)
Diluents and concentrations of silk fibroin aqueous solution and control sample: a silk fibroin aqueous solution and a control sample were diluted to from 1.0 mass/vol % to 1.5 mass/vol % and about 1.3 mass/vol %, respectively, with an 8 M urea aqueous solution.
Excitation wavelength: 630 nm
Detection wavelength: 680 nm

In the calculation of the molecular weight of silk fibroin, dedicated 2100 Expert software was used. The molecular weight of silk fibroin was calculated from a molecular weight calibration curve obtained from data on a molecular weight standard ladder measured together with the sample. The most intensely colored band was used as a band in electrophoresis to be used for the calculation of the molecular weight.

Method of evaluating Storage Stability

5 mL of the fibroin aqueous solution prepared in each of Examples and Comparative Examples described below was sealed in a glass vial, and was evaluated for storage stability in a refrigerator at 4° C. The evaluation was visually performed every day, and a time period until the fibroin aqueous solution formed gel and lost its fluidity was defined as a time period for which the solution can be stored. The evaluation was performed in comparison to the aqueous solutions (Comparative Examples 1 to 5), each of which is free of any additive and has the same molecular weight and the same concentration, based on the following criteria.
A: The aqueous solution can be stored longer than the additive-free product by 30 days or more.
B: The aqueous solution can be stored longer than the additive-free product by from 15 days to 29 days.
C: The aqueous solution can be stored longer than the additive-free product by from 5 days to 14 days.
D: The aqueous solution can be stored longer than the additive-free product by from 0 days to 4 days.
E: The aqueous solution can be stored shorter than the additive-free product.

When the evaluation rank was A or B, the storage stability was determined to be satisfactory.

### Method of Evaluating Foamability

The evaluation of foamability was performed by gel formation using a pH change with hydrochloric acid.

9 mL of a fibroin aqueous solution was loaded into a glass vial, and then 1 mL of 0.3 M hydrochloric acid was added dropwise thereto. The vial was gently shaken and left to stand still in an incubator at 37° C. The time period taken for gel formation was measured. The evaluation was performed in comparison to the aqueous solutions (Comparative Examples 1 to 5), each of which was free of any additive and had the same molecular weight and the same concentration, based on the following criteria.
A: The aqueous solution can form foam in a time period comparable to that of the additive-free product.
B: The time period taken for the aqueous solution to form foam is longer than that of the additive-free product by from 12 hours to 1 day.
C: The time period taken for the aqueous solution to form foam is longer than that of the additive-free product by from 2 days to 3 days.
D: The time period taken for the aqueous solution to form foam is longer than that of the additive-free product by 4 days or more.

When the evaluation rank was A or B, the foamability was determined to be satisfactory.

### Example 1

### (Refining Step)

4.5 L of ultrapure water was heated and boiled in a 5 L glass beaker, and then 8.48 g of sodium carbonate (manufactured by Kishida Chemical Co., Ltd.) was added thereto to provide a 0.02 mol/L sodium carbonate solution.

10 g of a cocoon of a domesticated silkworm (manufactured by Tajima Shoji Co., Ltd.) cut into about 1 cm squares was added to the sodium carbonate solution, and the mixture was heated for 30 minutes to provide fibroin having sericin removed therefrom. The fibroin was washed with cold ultrapure water. Then, the fibroin was drained, and was dried in a fume hood overnight to provide refined fibroin.

### (Neutral Salt Dissolving Step)

80.7 g of lithium bromide anhydride (manufactured by Kishida Chemical Co., Ltd.) was loaded into a measuring flask, and the volume was filled up to 100 mL with water. Thus, a 9.3 mol/L lithium bromide aqueous solution was obtained. 3.0 g of the refined fibroin was filled into a 100 mL glass beaker, and 14.8 mL of the 9.3 mol/L LiBr solution was added thereto so that the refined fibroin was completely immersed in the solution. The refined fibroin was dissolved in the solution in an oven at 60° C. for 2 hours to provide a transparent neutral salt aqueous solution.

### (Desalting Step)

19 mL of the neutral salt aqueous solution produced above was injected into a dialysis cassette (manufactured by Thermo Fisher Scientific Inc.) having a molecular weight cut-off of 3,500 and a capacity of 30 mL through use of a syringe, and dialysis was performed by immersing the dialysis cassette in 2 L of ultrapure water. The water was exchanged 1 hour after the start of the dialysis and 4 hours after that, and then the water was exchanged once every 8 hours. Thus, the dialysis was performed for a total of 53 hours to perform desalting. The resultant aqueous solution was centrifuged twice at 4° C. for 20 minutes at 11,000 rpm with a centrifuge CR7N (manufactured by Eppendorf Himac Technologies Co., Ltd.) to precipitate insoluble matter. Thus, a fibroin aqueous solution was obtained. The resultant fibroin aqueous solution had a solid content concentration of 8% and a molecular weight of 150 kDa.

### (Additive Adding Step and Concentration Adjusting Step)

0.85 g of a 7% tetramethylammonium chloride aqueous solution (7.4% with respect to fibroin) and 0.58 g of ultrapure water were added to 10 g of the above-mentioned fibroin aqueous solution, and the mixture was stirred with a mix rotor until the mixture became uniform to provide an aqueous solution of Example 1 having a fibroin concentration of 7%.

A storage stability test was performed on the aqueous solution of Example 1. As a result, it was found that the aqueous solution was able to be stored for 82 days. In addition, the aqueous solution was evaluated for foamability. As a result, it was found that gel was formed in 6 hours.

### Examples 2 to 11 and 16 to 23

Aqueous solutions of Examples 2 to 11 and 16 to 23 were each prepared by the same method as in Example 1 except that a compound shown in Table 1 was added instead of tetramethylammonium chloride in the additive adding step. The concentration and addition amount of the additive aqueous solution and the addition amount of ultrapure water were appropriately adjusted so that the fibroin concentration and the addition amount of the additive shown in Table 1 were achieved.

### Example 12

An aqueous solution of Example 12 was prepared by the same method as in Example 11 except that the heating time in the refining step was changed from 30 minutes to 10 minutes.

### Example 13

An aqueous solution of Example 13 was prepared by the same method as in Example 11 except that the heating time in the refining step was changed from 30 minutes to 120 minutes.

### Examples 14 and 15

Aqueous solutions of Examples 14 and 15 were each prepared by the same method as in Example 11 except that, after the desalting step, an aqueous solution concentrated in a concentration step described below was used and the fibroin concentration was set to 15% or 25% in the concentration adjusting step.

### (Concentration Step)

The fibroin aqueous solution (solid content concentration: 8%) after the desalting step was sealed in a dialysis tube (manufactured by Repligen Corporation) having a molecular weight cut-off of 10,000, and was concentrated for 10 hours under an environment at 10° C. and 5% RH with an airflow from a blower. The resultant fibroin aqueous solution had a solid content concentration of 28%.

### Comparative Examples 1 to 5

Aqueous solutions of Comparative Examples 1 to 5 were each prepared by the same method as in Examples 1 and 12 to 15 except that no additive was added in the additive adding step.

### Comparative Examples 6 to 8

Aqueous solutions of Comparative Examples 6 to 8 were each prepared by the same method as in Example 1 except that a compound shown in Table 1 was added instead of tetramethylammonium chloride in the additive adding step.

The evaluation results of the fibroin aqueous solutions prepared as described above are summarized in Table 1.

TABLE 1-1

| | | | | |
|---|---|---|---|---|
| Ex. No. | General | Compound name | —R¹ | —R² |
| Ex. 1 | 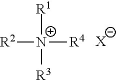 | Tetramethylammonium chloride | —CH₃ | —CH₃ |
| Ex. 2 | | Tetramethylammonium acetate | —CH₃ | —CH₃ |
| Ex. 3 | | Tetraethylammonium bromide | —CH₂CH₃ | —CH₂CH₃ |
| Ex. 4 | | Tetraethylammonium hydroxide | —CH₂CH₃ | —CH₂CH₃ |
| Ex. 5 | | Tetrapropylammonium chloride | —(CH₂)₂CH₃ | —(CH₂)₂CH₃ |
| Ex. 6 | | Tetrabutylammonium chloride | —(CH₂)₃CH₃ | —(CH₂)₃CH₃ |
| Ex. 7 | | Triethylmethylammonium | —CH₃ | —CH₂CH₃ |
| Ex. 8 | | Trimethyloctylammonium | —CH₃ | —CH₃ |
| Ex. 9 | | Trimethylphenylammonium | —CH₃ | —CH₃ |
| Ex. 10 | | Choline | —CH₃ | —CH₃ |
| Ex. 11 | | Choline chloride | —CH₃ | —CH₃ |
| Ex. 12 | | Choline chloride | —CH₃ | —CH₃ |
| Ex. 13 | | Choline chloride | —CH₃ | —CH₃ |
| Ex. 14 | | Choline chloride | —CH₃ | —CH₃ |
| Ex. 15 | | Choline chloride | —CH₃ | —CH₃ |
| Ex. 16 | | Choline bitartrate | —CH₃ | —CH₃ |
| Ex. 17 | | Trimethylamine hydrochloride | —CH₃ | —CH₃ |
| Ex. 18 | | Triethanolamine hydrochloride | —(CH₂)₂OH | —(CH₂)₂OH |
| Ex. 19 | | Dibutylamine hydrochloride | —(CH₂)₃CH₃ | —H |
| Ex. 20 | | Glycine ethyl ester | —CH₂CO₂CH₂CH₃ | —H |
| Ex. 21 | | Glycinamide hydrochloride | —CH₂CONH₂ | |

| | | | |
|---|---|---|---|
| | General | | Ring structure formed by |
| Ex. No. | formula | Compound name | C—N═C and A |
| Ex. 22 | 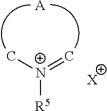 | Methylpyridinium chloride | Pyridinium |
| Ex. 23 | | 1,3-Dimethylimidazolium | 3-Dimethylimidazolium |
| | | chloride | |

Table 1-2

| | | | |
|---|---|---|---|
| Ex. No. | —R³ | —R⁴ | X⁻ |
| Ex. 1 | —CH₃ | —CH₃ | Cl⁻ |
| Ex. 2 | —CH₃ | —CH₃ | CH₃CO₂⁻ |
| Ex. 3 | —CH₂CH₃ | —CH₂CH₃ | Br⁻ |
| Ex. 4 | —CH₂CH₃ | —CH₂CH₃ | OH⁻ |
| Ex. 5 | —(CH₂)₂CH₃ | —(CH₂)₂CH₃ | Cl⁻ |
| Ex. 6 | —(CH₂)₃CH₃ | —(CH₂)₃CH₃ | Cl⁻ |
| Ex. 7 | —CH₂CH₃ | —CH₂CH₃ | Cl⁻ |
| Ex. 8 | —CH₃ | —(CH₂)₇CH₃ | Cl⁻ |
| Ex. 9 | —CH₃ | —C₆H₅ | Cl⁻ |
| Ex. 10 | —CH₃ | —CH₂CH₂OH | OH⁻ |
| Ex. 11 | —CH₃ | —CH₂CH₂OH | Cl⁻ |
| Ex. 12 | —CH₃ | —CH₂CH₂OH | Cl⁻ |
| Ex. 13 | —CH₃ | —CH₂CH₂OH | Cl⁻ |
| Ex.14 | —CH₃ | —CH₂CH₂OH | Cl⁻ |
| Ex. 15 | —CH₃ | —CH₂CH₂OH | Cl⁻ |
| Ex.16 | —CH₃ | —CH₂CH₂OH | (CHOH)₂(CO₂H)(CO₂)⁻ |
| Ex.17 | —CH₃ | —H | Cl⁻ |
| Ex. 18 | —(CH₂)₂OH | —H | Cl⁻ |
| Ex. 19 | —H | —H | Cl⁻ |
| Ex. 20 | —H | —H | Cl⁻ |
| Ex. 21 | —H | —H | Cl⁻ |
| | Ring structure formed | | |
| Ex. No. | by C—N═C and A | —R⁵ | X⁻ |
| Ex. 22 | Pyridinium | —CH₃ | —CI |
| Ex. 23 | 3-Dimethylimidazolium | —CH₃ | —CI |

TABLE 2

| | | Fibroin |
|---|---|---|
| Example No. | Compound name | concentration |
| Example 1 | Tetramethylammonium chloride | 7% |
| Example 2 | Tetramethylammonium acetate | 7% |
| Example 3 | Tetraethylammonium bromide | 7% |
| Example 4 | Tetraethylammonium hydroxide | 7% |
| Example 5 | Tetrapropylammonium chloride | 7% |
| Example 6 | Tetrabutylammonium chloride | 7% |
| Example 7 | Triethylmethylammonium chloride | 7% |
| Example 8 | Trimethyloctylammonium chloride | 7% |
| Example 9 | Trimethylphenylammonium | 7% |
| | chloride | |
| Example 10 | Choline | 7% |
| Example 11 | Choline chloride | 7% |
| Example 12 | Choline chloride | 7% |
| Example 13 | Choline chloride | 7% |
| Example 14 | Choline chloride | 15% |
| Example 15 | Choline chloride | 25% |
| Example 16 | Choline bitartrate | 7% |
| Example 17 | Trimethylamine hydrochloride | 7% |
| Example 18 | Triethanolamine hydrochloride | 7% |
| Example 19 | Dibutylamine hydrochloride | 7% |
| Example 20 | Glycine ethyl ester hydrochloride | 7% |
| Example 21 | Glycinamide hydrochloride | 7% |
| Example 22 | Methylpyridinium chloride | 7% |
| Example 23 | 1,3-Dimethylimidazolium chloride | 7% |
| Comparative Example 1 | No additive | 7% |
| Comparative Example 2 | No additive | 7% |
| Comparative Example 3 | No additive | 7% |
| Comparative Example 4 | No additive | 15% |
| Comparative Example 5 | No additive | 25% |
| Comparative Example 6 | Urea | 7% |
| Comparative Example 7 | Guanidine chloride | 7% |
| Comparative Example 8 | Arginine | 7% |

| | | Addition | | |
|---|---|---|---|---|
| | Molecular | amount of | Storage | |
| | weight | additive | stability | |
| Example No. | [kDa] | wt % | [4° C.] | Foamability |
| Example 1 | 150 | 7.4 | A | A |
| Example 2 | 150 | 9.0 | B | A |
| Example 3 | 150 | 14.1 | A | A |
| Example 4 | 150 | 6.1 | B | A |
| Example 5 | 150 | 14.9 | A | A |
| Example 6 | 150 | 18.7 | A | A |
| Example 7 | 150 | 10.2 | A | A |
| Example 8 | 150 | 14.0 | B | B |
| Example 9 | 150 | 11.5 | A | A |
| Example 10 | 150 | 8.1 | A | A |
| Example 11 | 150 | 9.4 | A | A |
| Example 12 | 230 | 9.4 | B | A |
| Example 13 | 110 | 9.4 | A | A |
| Example 14 | 150 | 9.4 | B | A |
| Example 15 | 150 | 9.4 | B | A |
| Example 16 | 150 | 17.0 | A | A |
| Example 17 | 150 | 6.4 | B | A |
| Example 18 | 150 | 12.5 | B | A |
| Example 19 | 150 | 11.1 | B | A |
| Example 20 | 150 | 9.4 | A | A |
| Example 21 | 150 | 5.0 | A | A |
| Example 22 | 150 | 8.7 | B | A |
| Example 23 | 150 | 8.9 | B | A |
| Comparative Example 1 | 150 | — | 34 days | 6 hours |
| Comparative Example 2 | 230 | — | 16 days | 3 hours |
| Comparative Example 3 | 110 | — | 69 days | 1 day |
| Comparative Example 4 | 150 | — | 17 days | 3 hours |
| Comparative Example 5 | 150 | — | 10 days | 2 hours |
| Comparative Example 6 | 150 | 4.0 | A | D |
| Comparative Example 7 | 150 | 6.4 | A | D |
| Comparative Example 8 | 150 | 11.7 | A | D |

As shown in Table 1, it is understood that, through incorporation of the compound represented by the general formula (1) or (2), an aqueous solution that is excellent in storage stability and is also excellent in foamability is obtained.

### Example 24

### (Method of Producing Fibroin Aqueous Solution Including Step of Removing Insoluble Matter)

An aqueous solution of Example 24 was prepared by the same method as in Example 1 except that, after the desalting step of Example 1, a heating step, a cooling step, and a microfiltration step were performed.

### (Heating, Cooling, and Microfiltration Steps)

The fibroin aqueous solution obtained through the desalting step of Example 1 was heated at 90° C. for 45 minutes, and was then rapidly cooled to 5° C. with ice water. The resultant aqueous solution was subjected to microfiltration with a membrane filter (pore diameter: 1 m, made of hydrophilic PTFE, manufactured by Merck Ltd.).

The fibroin aqueous solution obtained above was subjected to the additive adding step and the concentration adjusting step by the same method as in Example 1 to provide a fibroin aqueous solution of Example 24.

A storage stability test was performed on the aqueous solution of Example 24. As a result, it was found that the aqueous solution was able to be stored for 200 days or more. In addition, the aqueous solution was evaluated for foamability. As a result, it was found that gel was formed in 6 hours in the same manner as in Example 1 and Comparative Example 1.

### <Evaluation of Foamability by Sponge Formation>

The evaluation of foamability by sponge formation was performed through use of each of the fibroin aqueous solutions of Example 1, Comparative Example 1, and Comparative Example 6.

### Example 25

2 mL of the fibroin aqueous solution of Example 1 was loaded into a plastic vial, and 4 g of salt sieved to a particle diameter of from 500 μm to 750 m was slowly added thereto. The vial was lightly tapped so that air bubbles were removed. Then, the vial was left to stand in an incubator at 37° C. for 2 days. The resultant solid was left to stand still in 1 L of ultrapure water for half a day so that particles of the salt were removed. This process was repeated five times, and then the resultant porous body was air-dried to provide a fibroin sponge.

### Comparative Example 9

A fibroin sponge was formed by the same method as in Example 25 except that the fibroin aqueous solution of Comparative Example 1 was used. As a result, a fibroin sponge equivalent to that of Example 25 was obtained.

### Comparative Example 10

A fibroin sponge was formed by the same method as in Example 25 except that the fibroin aqueous solution of Comparative Example 6 was used. However, no fibroin sponge was obtained even after the fibroin aqueous solution was left to stand in the incubator for 10 days.

### <Evaluation of Foamability by Gel Formation Using Ultrasonic Wave>

The evaluation of foamability by gel formation using an ultrasonic wave was performed through use of each of the fibroin aqueous solutions of Example 1, Comparative Example 1, and Comparative Example 6.

### Example 26

5 mL of the fibroin aqueous solution of Example 1 was loaded into a 15 mL conical tube, and ultrasonic irradiation was performed for 1 minute with an ultrasonic homogenizer (manufactured by Tomy Kogyo Co., Ltd.). The aqueous solution was left to stand still in an incubator at 37° C., and the time period taken for gel formation was observed. As a result, fibroin gel was obtained 6 hours later.

### Comparative Example 11

Fibroin gel was formed by the same method as in Example 26 except that the fibroin aqueous solution of Comparative Example 1 was used. As a result, fibroin gel was obtained 6 hours later in the same manner as in Example 26.

### Comparative Example 12

Fibroin gel was formed by the same method as in Example 26 except that the fibroin aqueous solution of Comparative Example 6 was used. As a result, it took 7 days to obtain gel.

According to the present invention, a fibroin aqueous solution that is excellent in storage stability and is also excellent in foamability, and a method of producing the same can be provided.

According to the present invention, the fibroin aqueous solution that is excellent in long-term storage stability and can be easily formed into various types of foam can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A fibroin aqueous solution comprising:
fibroin; and
a compound represented by the following general formula (1) or (2): 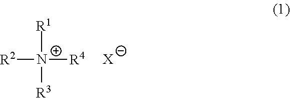
in the general formula (1),
R¹ to R⁴ each independently represent
a hydrogen atom,
an aliphatic hydrocarbon group having 1 or more and 8 or less carbon atoms, which may have a substituent, or
an aryl group or an aralkyl group having 6 or more and 10 or less carbon atoms, which may have a substituent,
provided that at least one of R¹ to R⁴ does not represent a hydrogen atom, and
X⁻ represents an anion; 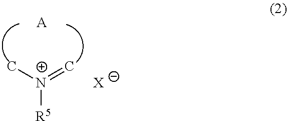
in the general formula (2),
C—N═C and A form a ring structure together,
R⁵ represents
a hydrogen atom,
an aliphatic hydrocarbon group having 1 or more and 8 or less carbon atoms, which may have a substituent, or
an aryl group or an aralkyl group having 6 or more and 10 or less carbon atoms, which may have a substituent, and
X⁻ represents an anion.

2. The fibroin aqueous solution according to claim 1,
wherein the fibroin aqueous solution comprises the compound represented by the general formula (1), and
wherein, in the general formula (1),
R¹ to R⁴ each independently represent
a hydrogen atom,
an alkyl group having 1 or more and 8 or less carbon atoms, which may have a hydroxy group, an amino group, an alkoxycarbonyl group, or a carbamoyl group as a substituent, or
an aryl group or an aralkyl group having 6 or more and 10 or less carbon atoms.

3. The fibroin aqueous solution according to claim 1,
wherein the fibroin aqueous solution comprises the compound represented by the general formula (2), and
wherein, in the general formula (2),
C—N═C and A form pyridine or imidazole together, and
R⁵ represents
a hydrogen atom,
an alkyl group having 1 or more and 8 or less carbon atoms, or
an aryl group or an aralkyl group having 6 or more and 10 or less carbon atoms.

4. The fibroin aqueous solution according to claim 1, wherein, in the general formula (1) or the general formula (2), X⁻ represents an anion selected from a halide ion, a hydroxide ion, and a carboxylate anion.

5. The fibroin aqueous solution according to claim 1,
wherein the fibroin aqueous solution comprises the compound represented by the general formula (1), and
wherein the compound represented by the general formula (1) is a halide, hydroxide, or carboxylate of any one selected from a tetraalkylammonium, choline, a choline derivative, glycine, and a glycine derivative.

6. The fibroin aqueous solution according to claim 1,
wherein the fibroin aqueous solution comprises the compound represented by the general formula (1), and
wherein the compound represented by the general formula (1) is any one selected from a tetraalkylammonium halide, and a halide, hydroxide, or carboxylate of any one selected from choline, a choline derivative, glycine, and a glycine derivative.

7. The fibroin aqueous solution according to claim 1, wherein the fibroin has a molecular weight of 100,000 or more.

8. The fibroin aqueous solution according to claim 1, wherein the fibroin has a concentration of from 5 mass % to 40 mass %.

9. The fibroin aqueous solution according to claim 1, wherein the fibroin is derived from silk.

10. A method of producing a fibroin aqueous solution comprising the steps of:
adding a compound represented by the following general formula (1) or (2) to a fibroin aqueous solution; and
adjusting a concentration of the obtained solution, 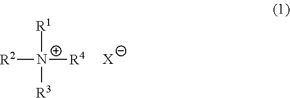
in general formula (1),
R¹ to R⁴ each independently represent
a hydrogen atom,
an aliphatic hydrocarbon group having 1 or more and 8 or less carbon atoms, which may have a substituent, or
an aryl group or an aralkyl group having 6 or more and 10 or less carbon atoms, which may have a substituent,
provided that at least one of R¹ to R⁴ does not represent a hydrogen atom, and
X⁻ represents an anion; 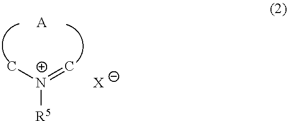
in the general formula (2),
C—N═C and A form a ring structure together,
R⁵ represents
a hydrogen atom,
an aliphatic hydrocarbon group having 1 or more and 8 or less carbon atoms, which may have a substituent, or
an aryl group or an aralkyl group having 6 or more and 10 or less carbon atoms, which may have a substituent, and
X⁻ represents an anion.

11. The method of producing a fibroin aqueous solution according to claim 10 further comprising:
a neutral salt dissolving step of dissolving a refined fibroin raw material in a neutral salt aqueous solution to provide a fibroin-neutral salt aqueous solution;
a desalting step of desalting the fibroin-neutral salt aqueous solution to provide a fibroin aqueous solution;
a heating step of heating the fibroin aqueous solution at 65° C. or more and 110° C. or less for a time period of 30 minutes or more and 60 minutes or less;
a cooling step of rapidly cooling the fibroin aqueous solution heated in the heating step to 15° C. or less; and
a microfiltration step of filtering the fibroin aqueous solution cooled in the cooling step through use of a microfiltration membrane.

12. An article comprising:
fibroin; and
a compound represented by the following general formula (1) or (2): 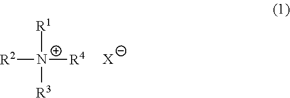
in the general formula (1),
R¹ to R⁴ each independently represent
a hydrogen atom,
an aliphatic hydrocarbon group having 1 or more and 8 or less carbon atoms, which may have a substituent, or
an aryl group or an aralkyl group having 6 or more and 10 or less carbon atoms, which may have a substituent,
provided that at least one of R¹ to R⁴ does not represent a hydrogen atom, and
X⁻ represents an anion; 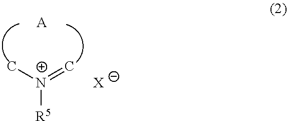
in the general formula (2),
C—N═C and A form a ring structure together,
R⁵ represents
a hydrogen atom,
an aliphatic hydrocarbon group having 1 or more and 8 or less carbon atoms, which may have a substituent, or
an aryl group or an aralkyl group having 6 or more and 10 or less carbon atoms, which may have a substituent, and
X⁻ represents an anion.

13. The article according to claim 12, wherein the article has any one of powder, film, sponge, and gel shapes, or is a molded body that is molded with a mold.

14. The article according to claim 12, wherein the fibroin is derived from silk.
